(19) United States
(12) Patent Application Publication
(10) Pub. No.: US 2015/0366941 A1
(43) Pub. Date: Dec. 24, 2015

(54) HUMAN DISEASE TREATMENT WITH MEDICAL FOOD FORMULATION

(71) Applicants: John Edgar Menear, Santa Cruz, CA (US); Joseph Ramaekers, Aptos, CA (US)

(72) Inventors: John Edgar Menear, Santa Cruz, CA (US); Joseph Ramaekers, Aptos, CA (US)

(73) Assignee: CortControl, Santa Cruz, CA (US)

(21) Appl. No.: 14/738,724

(22) Filed: Jun. 12, 2015

Related U.S. Application Data
(60) Provisional application No. 61/998,139, filed on Jun. 19, 2014.

(51) Int. Cl. A61K 38/17; A61K 31/716; A23L 1/30; A61K 8/64; A61K 8/73; A61Q 19/08; A61K 35/74; A61K 8/99

(52) U.S. Cl. CPC A61K 38/1709; A61K 35/74; A61K 31/716; A61K 8/99; A61K 8/64; A61K 8/73; A61Q 19/08; A23L 1/3002; A23L 1/3014; A23V 2002/00

(57) ABSTRACT

A method of using a medical food containing at least transfer factor and lactic acid generating bacteria to mitigate the symptoms of a specific human disease. Another medical food formulation consists of transfer factor, lactic acid generating bacteria, and glucans in appropriate combinations. Other components may be added. The medical food, administered correctly, reduces cortisol levels, builds the immune system, and balances the endocrine system. Dosage amounts are adjusted for client weight. Medical foods may be used with other treatment options.

### CROSS-REFERENCE TO RELATED APPLICATIONS

This application claims priority to U.S. Provisional Application 61/998,139 filed Jun. 19, 2014, which is hereby incorporated by reference in its entirety.

### FIELD OF THE INVENTION

The invention relates to a method of administering medical food compositions and formulations comprising at least transfer factor and lactic acid generating bacteria to support treatment for several conditions. In some embodiments, glucans are added to the transfer factor and lactic acid generating bacteria. In particular, the conditions addressed with the medical food include (1) early stage Alzheimers, (2) upper respiratory infections, (3) lower respiratory infections, (4) osteoarthritis, (5) osteopenia, (6) osteoporosis and bone weakness (7) proteolysis, (8) muscle wasting, (9) early stage adrenal fatigue, (10) early stage chronic fatigue syndrome, (11) psychological stress, (12) addiction, (13) cancer (various forms), (14) diarrheal disease, and (15) cognitive deficits due to the brain's ability to utilize glucose, (16) skin aging, (17) hypertension, (18) viral infections, (19) irritable bowel syndrome, (20) storage of abdominal fat, (21) multiple sclerosis, (22) anorexia nervosa, (23) candida, (24) intestinal parasites, (25) suboptimal testosterone levels, (26) AIDS, (27) viral hepatitis, (28) weight gain, or (29) arthropenia (loss of joint cartilage).

### BACKGROUND OF THE INVENTION

Veterinary field experience has shown that a combination of transfer factor and lactic acid generating bacteria is useful to address a variety of pet and livestock ailments. Adding glucans to the combination provides nearly a two-fold synergistic effect.

The combination of (1) transfer factor and lactic acid generating bacteria or (2) transfer factor, lactic acid generating bacteria, and glucans is normally administered as a food for a variety of mammals. However, other forms of administration are acceptable.

Success with animal ailments is being adopted for human ailments. Anecdotal human results have demonstrated the benefit of treating humans with the same medical food used for other mammals.

One important benefit of the employed medical food is cortisol reduction, which is described more below. However, cortisol reduction is not the only functional effect of the medical food. No healing mechanisms are proposed, but results indicate that cortisol reduction is not acting alone.

The employed medical food also enhances the immune system, and appears to rebalance the endocrine system. Digestive improvement is also involved, which affects overall mammal health.

This application is not limited by any proposed healing mechanism. The results are based on success with animal, not theory. The inventors know that the medical food works, but the underlying reasons are not fully understood.

Cortisol is found in higher-than-normal levels in diseases ranging from AIDS and Multiple Sclerosis to Alzheimer's and heart disease. Prolonged high levels of cortisol can throw the immune system into chaos and ravage the human body. A growing number of researchers believe that many of the worst, and least-understood, diseases will soon be identified as caused (at least partly) by persistently high cortisol.

Published studies have linked high cortisol to the conditions cited in the FIELD OF THE INVENTION above. The cortisol interaction varies. But high cortisol remains a correlating factor.

The veterinary field led the way, and developed hard data on food-based solutions for diseases. Medical food compositions based on (1) transfer factor and lactic acid generating bacteria or (2) transfer factor, lactic acid generating bacteria, and glucans were shown to be highly effective for livestock ad pet sicknesses.

In 2006, a discovery was made that these same food compositions lowered cortisol levels in cattle and other mammals. It was later noted that food utilization improved, skin and fur quality improved, weight gains occurred, joint function improved, muscle mass benefited, and energy levels were better. These observations support the use of the medical food for the disease categories cited in the FIELD OF THE INVENTION.

The experience of livestock is significant because the hormonal mechanism of stress is the same in all mammals. Humans generate the stress hormone, cortisol, in the same way as a goat, pig, cow, horse, or monkey. Basically, the pituitary releases ACTH (adrenocorticotropic hormone). Then ACTH stimulates the adrenal cortex to secrete cortisol.

Immunity enhancement also has similarities across species. For example, transfer factor from a cow can be used to improve immune response of a human. And good digestion is important across species.

Cortisol has a positive value in the short term. It energizes the body for a fight or flight situation. But a cortisol excess over a long time is destructive. Human health is improved by achieving baseline cortisol levels that fall within a normal concentration range.

For example, a negative effect on bone density due to the cortisol excess exists. By rebalancing cortisol (and associated endocrine functions), bone deposition increases and bone loss decreases. Mechanisms such as the calcium malabsorption, hypercalciuria and hypogonadism may apply. Cortisol can mobilize calcium from our bones, and circulate it back into our blood stream. Hypercortisolism, also called Cushing's syndrome, leads to osteoporosis and fractures in up to the 70% of cases, even in the presence of normal gonadal status.

From the viewpoint of Alzheimer's disease, there is new evidence that cortisol can cause damage to brain cells, especially in the hippocampus. New memories are made in the hippocampus. Cells in the hippocampus also serve as a negative feedback circuit to cortisol production. A vicious cycle ensues. As memories become harder to create, the production of cortisol simultaneously increases, more brain cells are lost, more cortisol is produced, and memories become even harder to create.

In addition, cortisol appears to hamper brain cell ability to utilize glucose.

Cortisol affects the digestive tract by killing the friendly bacteria and flora. If someone has irritable bowel syndrome, there's a good chance that cortisol levels are elevated. Intestinal parasites and Candida are likely to thrive in the presence of high cortisol.

High cortisol affects personal appearance. Elevated cortisol reduces synthesis of hyaluronan and proteoglycans, and contributes to their faster degradation. Hyaluronan and proteoglycans are responsible for hydrating the skin; they attract and hold adequate amounts of moisture. Without them, skin develops a dry inelastic quality.

High cortisol lowers testosterone levels and sperm count in males. Libido is reduced in both sexes. Tiredness and fatigue are accompanying symptoms.

Mechanisms are provided above to explain the cortisol effects with examples. However, this application is not limited by any proposed mechanism. The core information is that high cortisol correlates to various diseases.

Human information can be extrapolated from mammal studies. All mammals have a cortisol producing glandular system that mimics humans. Basically, the pituitary gland secretes ACTH (adrenocorticotropic hormone), which then stimulates the adrenal glands to release cortisol. Success with mammals is transferrable to humans.

Cattle studies have repeatedly demonstrated that feeding a mixture of transfer factor and lactic acid generating bacteria facilitates health. Glucans are often added to synergistically increase the immune response.

Medical technology addresses disease with a variety of treatments. Some of these treatments may have side effects. In contrast, the mixture of (1) transfer factor and lactic acid generating bacteria or (2) transfer factor, lactic acid generating bacteria and glucans is a food, not a drug. Foods take longer to confer benefits than drugs. Depending on disease severity, medical food consumption confers benefits in three days to ninety days. In turn, medical foods comprising transfer factor, lactic acid generating bacteria and glucans are inherently safe.

The consuming public now understands that foods possess more than basic nutrition (protein, carbohydrate, fat, etc.). For example, 95% of consumers agree that “certain foods have health benefits that go beyond basic nutrition and may reduce the risk of disease or other health concerns”. More than 50% of consumers believe that foods can replace the use of drugs.

The Federal Drug Administration acknowledges this trend with the relatively new category of “Medical Foods”. Medical Foods should be supervised and monitored by a doctor, nutritionist, nurse, medical technician or equivalent health care professional.

A food-based treatment option is needed that addresses root causes, and avoids side effects. A food based treatment may be used in conjunction with other treatments.

### BRIEF SUMMARY OF THE INVENTION

This instant invention is a method of treating multiple diseases and clinical indications with foods that reduce human cortisol levels, build immunity, and balance endocrine function (among other functional effects). Areas of application include (1) early stage Alzheimers, (2) upper respiratory infections, (3) lower respiratory infections, (4) osteoarthritis, (5) osteopenia, (6) osteoporosis and bone weakness (7) proteolysis, (8) muscle wasting, (9) early stage adrenal fatigue, (10) early stage chronic fatigue syndrome, (11) psychological stress, (12) addiction, (13) cancer (various forms), (14) diarrheal disease, and (15) cognitive deficits due to the brain's ability to utilize glucose, (16) skin aging, (17) hypertension, (18) viral infections, (19) irritable bowel syndrome, (20) storage of abdominal fat, (21) multiple sclerosis, (22) anorexia nervosa, (23) candida, (24) intestinal parasites, (25) suboptimal testosterone levels, (26) AIDS, (27) viral hepatitis, (28) weight gain, or (29) arthropenia (loss of joint cartilage).

Following is a condensed summary of the invention. By necessity, details are omitted in order to simply state the essence of the invention. Omitted details within this section should not be construed in a way that limits or alters the scope of the invention.

One preferred medical food composition is a mixture of transfer factor and lactic acid generating bacteria. This composition is described in U.S. Pat. No. 6,962,718 issued to Joseph Ramaekers.

Another preferred food composition is a mixture of transfer factor, lactic acid generating bacteria, and glucans. Glucans may be present as mushrooms. This composition is encompassed by domineering U.S. Pat. No. 6,962,718, but contains one additional component (glucans).

Either preferred composition may be augmented with additional additives. Those additives may differ depending on the disease condition addressed. But either (1) transfer factor and lactic acid generating bacteria or (2) transfer factor, lactic acid generating bacteria and glucans are present in all medical food formulations that fall within the scope of this invention. typically consume higher dosages.

The relative proportion of transfer factor, lactic acid generating bacteria, and/or glucans within a dose may vary. Although typical proportions can be recited, proportions may be modified to best serve each individual.

For some clients, transfer factor, lactic acid generating bacteria, and glucans are taken together. For other clients, transfer factor, lactic acid generating bacteria, and glucans are taken at different times during the day or week. Component separation and consumption of each component at different times are within a two day period are covered by the scope of this invention.

Typical method-of-use steps include some or all of the following: (1) deciding that one of the conditions in the FIELD OF THE INVENTION may be an issue, (2) selecting the medical food dosage level, (3) consuming the medical food, (4) continuing medical food consumption until symptoms are reduced, (5) measuring hormonal levels (for example, cortisol) over the treatment period, and (6) adjusting dosage levels as appropriate.

Medical food consumption may be used alone or used with other treatments. If pharmaceutical drugs are employed in a treatment regimen, the health care professional may elect to continue the pharmaceutical drugs along with the medical food.

Transfer of successes from livestock disease to human disease is supported by several facts. First, cortisol biochemistry is the same among mammal species. Second, transfer factor functions the same across species. Third, lactic acid generating bacteria improve digestive health across the mammal spectrum. Forth, both transfer factor and glucans benefit the immune response of most mammals.

Human benefits have been observed, but have been difficult to document. Pet owners have admitted to buying the medical food for a pet, but then taking it themselves. Results have been positive. Some owners continue to purchase the medical foods long after the pet died.

Informal studies also suggest the connection between the medical food and human performance. Feeding the medical food to stressed athletes lowers the symptoms of cortisol excess. But more than cortisol is involved. With the medical food, athletes tend to get a better night's sleep, and perform better during events. College students consume these foods before final examinations, sleep better, and get better grades. Golfers use these foods to lower scores. Elite military squads are currently evaluating mission recovery.

A cortisol-to-disease correlation in humans and other mammals is supported by a preponderance of data. Lowered cortisol measurements correlate to consuming transfer factor, lactic acid generating bacteria, and/or glucans. Other stress hormones may also be lowered by consuming transfer factor, lactic acid generating bacteria, and/or glucans.

In humans, better skin quality plus improved flexibility and movement are noticeable outcomes.

### BRIEF DESCRIPTION OF THE DRAWINGS

FIG. 1 is a diagram of cortisol generation in mammals. Cortisol is cited as important because it is a primary stress hormone. But cortisol reduction does not fully explain the benefits of the employed medical food.

FIG. 2 shows a drop in cortisol for calves that consumed the medical food, relative to control calves that received a placebo. Cortisol reduction played an important part in reviving their health because the calves were considered stressed from shipment.

FIG. 3 shows an increase in insulin for test calves. Under the test conditions, the increased insulin translates into better food utilization. Food utilization is quantified by calf weight gain (see FIG. 4). Note that the insulin increase began on the seventh consumption day, and the positive effects continued for at least 60 days.

FIG. 4 shows the daily average weight gain of calves receiving medical food. Measurements were made at 12 and 60 days. The weight gain was categorized by veterinary doctors to be largely muscle, bone, and ligaments. This is the opposite of bone loss, muscle wasting, osteopenia, osteoporosis, bone weakness, proteolysis, or arthropenia (loss of joint cartilage).

FIG. 5 shows a drop in morbidity and mortality of cattle for a first herd, which unmistakably signals better health. Improved health is reflected in reduced symptoms for the conditions cited in the FIELD OF THE INVENTION. Morbidity and mortality were largely traceable to respiratory infections, diarrheal disease, hypertension, viral infections, candida, and intestinal parasites.

FIG. 6 shows a drop in morbidity and mortality of cattle for a second herd. Again, results of medical food administration are significant.

### DETAILED DESCRIPTION OF THE INVENTION

Transfer factor is produced by leucocytes and lymphocytes. Transfer factor comprises small water soluble polypeptides of about 44 amino acids that stimulate or transfer cell mediated immunity from one individual to another.

The properties, characteristics and processes for obtaining transfer factor or transfer factors are discussed in U.S. Pat. Nos. 4,816,563; 5,080,895; 5,840,700, 5,883,224 and 6,468,534, the contents of which are hereby incorporated by reference into the present application.

Alternative sources of transfer factor include avian transfer factor, ova transfer factor, and colostrum from goats, pigs, horses and humans. This listing is not complete. In addition, pooled combinations of transfer factors from any number of sources may be used.

In certain embodiments, substantially purified transfer factor has a molecular weight of less than 10,000 Daltons.

Transfer factor is commercially available, and known to be safe.

Lactic acid generating bacteria is an important component of the pertinent medical foods, and is GRAS (generally recognized as safe). Lactic acid generating bacteria support digestion and brain health. Lactic acid generating bacteria provide healthful effects that are found in non-pasteurized sauer kraut and cod liver oil. Within the intestinal tract, lactic acid generating bacteria are beneficial. It has been estimated that 80% of human health depends on beneficial intestinal bacteria.

A human body becomes stressed by poor digestion. Poor digestion contributes to high cortisol levels, intestinal parasitic buildup, weakened bones, muscle wasting, skin aging, early stage Alzheimers, upper respiratory infections, lower respiratory infections, osteoarthritis, osteopenia, osteoporosis, proteolysis, early stage adrenal fatigue, early stage chronic fatigue syndrome, psychological stress, addiction, cancer (various forms), diarrheal disease, cognitive deficits, hypertension, viral infections, irritable bowel syndrome, storage of abdominal fat, multiple sclerosis, anorexia nervosa, candida, suboptimal testosterone levels, AIDS, viral hepatitis, weight gain, and arthropenia (loss of joint cartilage). Lactic acid generating bacteria help reduce cortisol and increase the immune system via improved digestion.

Glucans (polysaccharides) are known to support the immune system. When combined with transfer factor and lactic acid generating bacteria, a synergy is created. The combined effect on health is greater than the effect predicted from summing the individual components. That synergy is dramatic—approximately two-fold.

Once a disease is diagnosed, the treatment should address the underlying causes. Elevated cortisol, compromised immunity, and imbalanced endocrine function are often among the underlying causes.

Most likely medicines will be prescribed, but medicines can have undesirable side effects.

A preferred food composition to treat the conditions cited in the instant FIELD OF THE INVENTION includes a mixture of transfer factor and lactic acid generating bacteria. This composition is patented (Joe Ramaekers, U.S. Pat. No. 6,962,718, claim 6, issued Nov. 8, 2005). The method of using transfer factor and lactic acid generating bacteria for treating diseases can be viewed as an improvement limitation on the existing commonly-owned composition patent. The improvement comprises the method of using the patented composition to improve a disease condition. An improvement claim is presented in the claims section.

Another preferred food composition is a mixture of transfer factor, lactic acid generating bacteria, and glucans. Glucans may be present as mushrooms. The method of using transfer factor, lactic acid generating bacteria, and glucans for treating specific diseases can also be viewed as an improvement to U.S. Pat. No. 6,962,718.

Addition of glucans to transfer factor and lactic acid generating bacteria creates a measureable immunity synergy that was not expected. Field tests showed that transfer factor plus lactic acid generating bacteria increased killer T cells by 250%, and glucans increased killer T cells by 40%. But the combination of transfer factor, lactic acid generating bacteria and glucans increased killer T cells by 500%.

The required components—(1) transfer factor and lactic acid generating bacteria or (2) transfer factor, lactic acid generating bacteria and glucans—may be augmented with additional additives. Chosen additives vary with the intended method of use. Example additives are minerals, probiotics, prebiotics, dimethyl glycine, ascorbic acid, Vitamin A, Vitamin D3, Vitamin E, Vitamin B1, Vitamin B2, Vitamin B12, dipotassium phosphate, potassium chloride, magnesium sulfate, calcium pantothenate, minerals, antioxidants, amino acids, nutraceuticals, inositol hexaphosphate (Ip6), mannans, olive leaf extract, and phytosterols. In certain preferred embodiments, mannans are derived from Aloe vera. In certain preferred embodiments, phytosterols may be derived from soya bean.

Probiotics additives include, but are not limited to B. subtilis, B. longum, B. thermophilium, B. coagulans, E. faecium, and S. cerevisia, L. casei, L. plantarum, Pediococcus acidilactici, Kluyveromyces marxianus fragillis and combinations thereof.

The above listings do not include all possible additives. The food compositions may also include one or more of the following: carrier proteins such as serum albumin; buffers such as sodium acetate; fillers such as microcrystalline cellulose, lactose, corn and other starches; binding agents; sweeteners and other flavoring agents; coloring agents; and polyethylene glycol. Additives are well known in the art, and are used in a variety of formulations.

The relative proportion of transfer factor, lactic acid generating bacteria, and glucans within the composition may vary widely.

However, some reasonable weight ranges for transfer factor are 0.05-50 mg/pound of body weight. Reasonable weight ranges for lactic acid generating bacteria are 0.47-10 mg/pound of body weight. This is based on a nominal live count of 2.5×10⁶ CFU/Ounce. Reasonable weight ranges for glucans are 0.1-10 mg/pound of body weight.

The method of using the medical foods may have some or all of the following steps:
(1) determine with measurement that cortisol, immune function, or endocrine balance is a correlating factor to a human's specific disease,
(2) select the correct proportion of transfer factor, lactic acid generating bacteria, and glucans,
(3) choose the correct dosage level,
(4) select a feeding frequency between five times per day and once per week,
(5) begin consumption,
(6) periodically adjust food dosage, and
(7) continue consumption until a positive outcome is achieved.

A preponderance of test data supports the effect of administering transfer factor, lactic acid generating bacteria, and/or glucans to overcome livestock diseases. Animal data is not pursued in depth herein because this instant application is focused on human diseases. However, humans are mammals, and livestock data is useful.

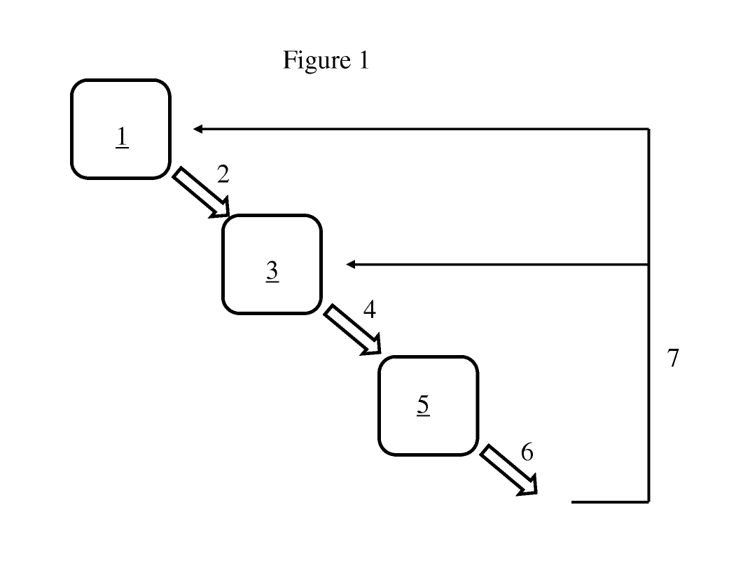

FIG. 1 shows the cortisol production sequence. FIG. 1 applies to humans and other mammals. The hypothalamus 1 releases corticotropin releasing hormone 2, which causes the anterior pituitary gland 3 to secrete adrenocorticotropic hormone 4. Adrenocorticotropic hormone 4 travels to the adrenal cortex 5, which responds by producing cortisol 6. Levels of cortisol are controlled by negative feedback loops 7.

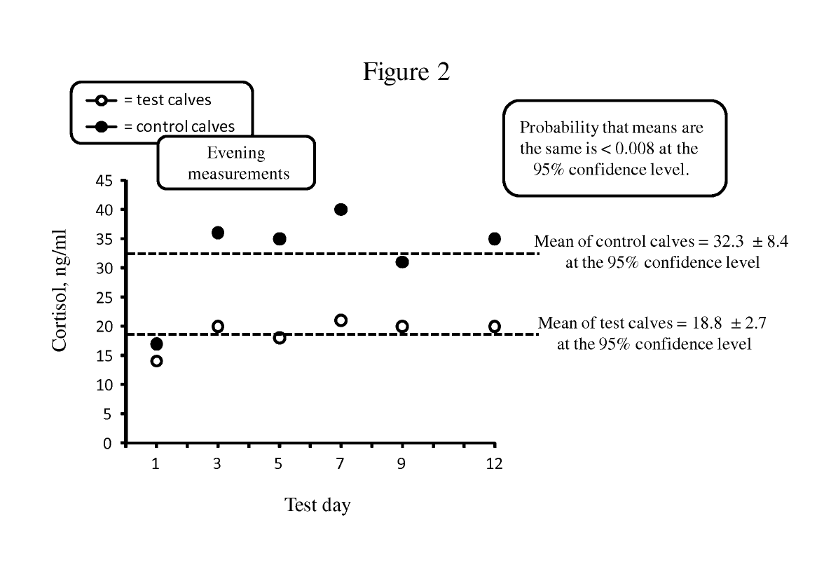

FIG. 2 graphically shows a drop in evening cortisol for stressed calves when fed a mixture of transfer factor, lactic acid generating bacteria, and/or glucans. This graph is provided to show how cortisol is decreased in mammals by feeding the appropriate medical food.

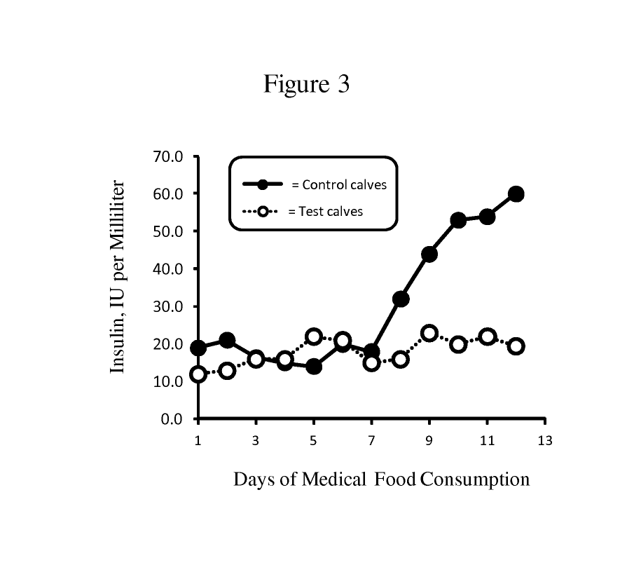

FIG. 3 shows how medical food affected insulin levels in cattle when an increase was desired. This shows that improvements are not limited to cortisol reduction alone. Other hormones are affected. As the calves became healthier, food utilization improved. This was detected as an insulin level increase. Note that it required 7 days to see this effect, and the effect continued beyond 12 days.

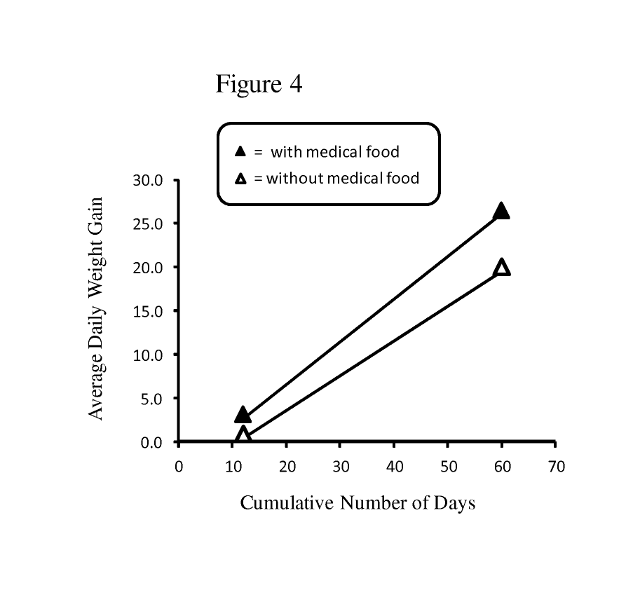

FIG. 4 is associated with FIG. 3. FIG. 4 graphically shows daily calf weight gain over a 60 day period. The weight gain correlated with the increased insulin levels in FIG. 3.

Calf weight gain supports the application of medical food for soft tissue and bone health. Veterinary observation indicated that the weight gain was largely from bone, muscle, and ligaments. Any hints of osteoarthritis, osteopenia, osteoporosis, arthropenia, muscle wasting, or bone weakness were being reversed. Skin quality and fur appearance were noticeably better.

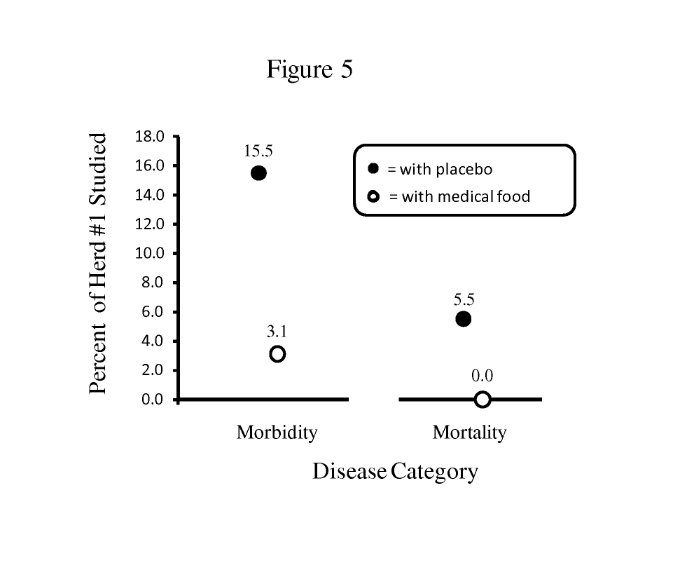

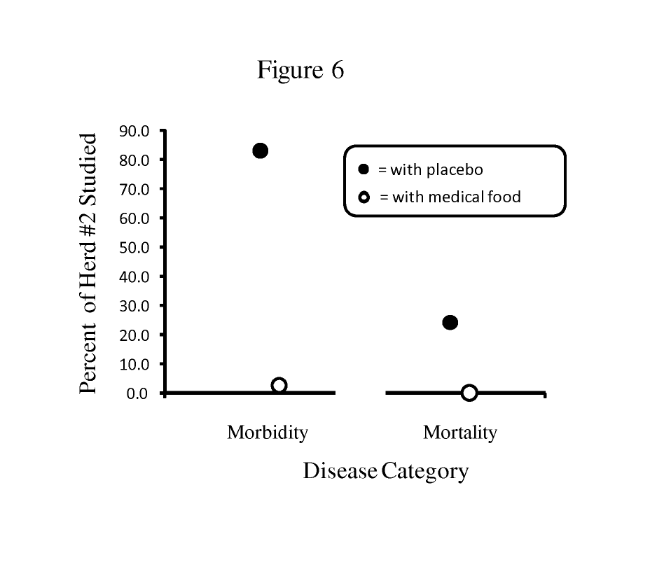

FIG. 5 and FIG. 6 show a drop in both morbidity and mortality for two different infected cattle herds. In both cases, viral and parasitic infections were involved. Respiration was affected. Diarrhea was common. Antibiotics were tried, but the herds remained sick. In contrast, the medical food solved the problem. This demonstrates the value of medical food for upper respiratory infections, lower respiratory infections, diarrheal disease, viral infections, irritable bowel syndrome, candida, intestinal parasites, AIDS, and viral hepatitis.

Performance data substantiate the value of medical food for early stage adrenal fatigue, early stage chronic fatigue syndrome, psychological stress, addiction, and hypertension. For example, race horses show marked improvement when medical food is added to the diet. Unpublished CortControl research shows special military forces recover faster from stressful missions. Golfers shoot lower scores after consuming medical food. Verbal reports describe how improved performance evolves from a less stressed mindset.

Several stress hormones and chemical markers benefit from a mixture of transfer factor, lactic acid generating bacteria, and/or glucans. Examples include alpha amalyase and T4 measurements of thyroid function.

Again, this application is based on actual observations—not theory. Although chemical markers and mechanisms are discussed, the scope of this application is not limited by and proposed mechanisms.

The invention claimed is:

1. A food-based method for treating human disease, comprising:
employing transfer factor transfer and lactic acid generating bacteria to create a medical food, wherein
said transfer factor includes some polypeptides with a molecular weight below 10,000 Daltons, and
said transfer factor is derived from avian, ova or colostrum sources;
feeding said medical food to said human, wherein
a dosage level is chosen based on said human's weight, and
the frequency of said feeding is between five times per day and once per week;
and
continuing said feeding until disease symptoms are reduced.

2. The food-based method of claim 1 wherein said human disease includes any one selected from a group including (a) early stage Alzheimers, (b) upper respiratory infections, (c) lower respiratory infections, (d) osteoarthritis, (e) osteopenia, (f) osteoporosis and bone weakness (g) proteolysis, (h) muscle wasting, (i) early stage adrenal fatigue, (j) early stage chronic fatigue syndrome, (k) psychological stress, (l) addiction, (m) cancer, (n) diarrheal disease, (o) cognitive deficits due to the brain's ability to utilize glucose, (p) skin aging, (q) hypertension, (r) viral infections, (s) irritable bowel syndrome, (t) storage of abdominal fat, (u) multiple sclerosis, (v) anorexia nervosa, (w) candida, (x) intestinal parasites, (y) suboptimal testosterone levels, (z) AIDS, (aa) viral hepatitis, (ab) weight gain, and (ac) arthropenia.

3. The food-based method of claim 1 wherein glucans are added to said medical food.

4. The food-based method of claim 3 wherein said glucans are derived from natural or hybrid mushrooms.

5. The food-based method of claim 1 further comprising measuring hormone levels periodically.

6. The food-based method of claim 5 wherein said hormone comprises cortisol.

7. The food-based method of claim 5 further comprising adjusting said dosage levels based on said measuring.

8. The food-based method of claim 1 wherein said transfer factor in each said dosage is present at 0.05 to 50 mg per pound of human body weight.

9. The food-based method of claim 1 wherein said lactic acid generating bacteria in each said dosage is present at 0.47 to 10 mg per pound of human body weight.

10. The food-based method of claim 9 wherein said lactic acid generating bacteria has a live count of 2.5 million colony forming units per ounce before inclusion into said medical food.

11. The food-based method of claim 3 wherein said glucans in each said dosage are present at 0.1 to 10 mg per pound of human body weight.

12. The food-based method of claim 1 wherein said continuing step requires between 3 days and 90 days.

13. The food-based method of claim 3 wherein transfer factor, lactic acid generating bacteria, and glucans are consumed at different times within a two-day period.

14. The food-based method of claim 3 wherein said human exhibits high baseline cortisol levels.

15. An improvement upon U.S. Pat. No. 6,962,718, claim 6 (issued Nov. 8, 2005 to Joseph Ramaekers, a current inventor) wherein the known part is
a medical food comprising pharmaceutically acceptable transfer factor and a pharmaceutically acceptable lactic acid generating bacteria wherein the amount of said transfer factor is from 10 mg to 10,000 mg per ounce of formulation; and
wherein the improvement is a method of using said medical food for treating human disease, comprising:
feeding said medical food to said human, wherein
a dosage level is chosen based on said human's weight, and
the frequency of said feeding is between five times per day and once per week;
and
continuing said feeding until disease symptoms are reduced.

16. The improvement in claim 15, wherein said human disease includes any one selected from a group including (a) early stage Alzheimers, (b) upper respiratory infections, (c) lower respiratory infections, (d) osteoarthritis, (e) osteopenia, (f) osteoporosis and bone weakness (g) proteolysis, (h) muscle wasting, (i) early stage adrenal fatigue, (j) early stage chronic fatigue syndrome, (k) psychological stress, (1) addiction, (m) cancer, (n) diarrheal disease, (o) cognitive deficits due to the brain's ability to utilize glucose, (p) skin aging, (q) hypertension, (r) viral infections, (s) irritable bowel syndrome, (t) storage of abdominal fat, (u) multiple sclerosis, (v) anorexia nervosa, (w) candida, (x) intestinal parasites, (y) suboptimal testosterone levels, (z) AIDS, (aa) viral hepatitis, (ab) weight gain, and (ac) arthropenia.

17. The improvement in claim 15, further comprising at least one step selected from a group including:
(a) adjusting proportions of transfer factor transfer and said lactic acid generating bacteria within said formulation to create a specific disease medical food supplement,
(b) measuring said patient's cortisol levels periodically,
(c) adjusting said dosage levels based on periodic cortisol measurements, and
(d) monitoring multiple stress hormones.

18. The improvement claim in claim 15, wherein glucans are added to said formulation.

19. The improvement claim in claim 18, wherein said glucans are present between at 0.1 to 10 mg per pound of patient's body weight.

20. The improvement claim in claim 15, wherein said lactic acid generating bacteria in each said dosage is present at 0.47 to 10 mg per pound of human body weight.

21. The improvement claim in claim 15 wherein said unmixed lactic acid generating bacteria has a live count of 2.5 million colony forming units per ounce.
